(19) United States
(12) Patent Application Publication
(10) Pub. No.: US 2026/0007513 A1
(43) Pub. Date: Jan. 8, 2026

(54) SYSTEMS AND METHODS FOR OPTIMIZING BLOOD FLOW

(71) Applicant: 4C Medical Technologies, Inc., Maple Grove, MN (US)

(72) Inventors: Janson Lee Ayer, Maple Grove, MN (US); Thomas Benson, Minneapolis, MN (US); Jonathan Berndt, Crystal, MN (US); Shelby Jo Byron, Maple Grove, MN (US); Jason S. Diedering, Minneapolis, MN (US); Olivia Johnson, Minneapolis, MN (US); Sounthara Khouengboua, Chaska, MN (US); Paul Thompson, Minnetonka, MN (US); Jaqueline Wendel, Hopkins, MN (US)

(21) Appl. No.: 19/066,444

(22) Filed: Feb. 28, 2025

Related U.S. Application Data
(60) Provisional application No. 63/559,987, filed on Mar. 1, 2024.

(51) Int. Cl. A61F 2/24

(52) U.S. Cl. CPC A61F 2/2418; A61F 2250/0036

(57) ABSTRACT

Apparatus and methods for an implant are provided. The implant may have a constrained configuration and a relaxed configuration. The implant may include an outer stent and an inner valve support. The inner valve support may be positioned within the outer stent. The implant may include a transition section spanning between the outer stent and the inner valve support.

### CROSS-REFERENCE TO RELATED APPLICATIONS

This application is a nonprovisional of U.S. Provisional Application No. 63/559,987, filed Mar. 1, 2024, which is hereby incorporated herein by reference in its entirety.

### BACKGROUND

The native heart valves, e.g., aortic, pulmonary, tricuspid and mitral valves, provide forward-only flow of an adequate supply of blood through the cardiovascular system. Native heart valves may lose functionality over time. Early interventions repaired or displaced dysfunctional valve(s) with the use of open-heart surgery. More recently, gaining access to the valve of interest has been achieved percutaneously via one of at least the following known access routes: transapical, transjugular, transfemoral; transatrial; and transseptal delivery techniques, collectively known as transcatheter techniques.

Typically, in a transcatheter technique, a prosthetic valve is mounted within a stented frame that is capable of achieving collapsed and expanded states. The frame is collapsed and advanced through a sheath or delivery catheter positioned in a blood vessel of the patient until reaching the delivery site. The stented frame is generally released from the catheter or sheath and, by a variety of means, expanded with the valve to a functional size and positioned in the heart in a functional orientation.

It therefore would be desirable to provide apparatus and methods for one or more of engaging, loading, translating, delivering, repositioning, resheathing and deploying an expandable stent to, and within, a heart chamber.

### BRIEF DESCRIPTION OF THE DRAWINGS

FIG. 1 illustrates an exemplary implant in accordance with the invention.

FIG. 2 illustrates an exemplary implant in accordance with the invention.

FIG. 3 illustrates a bottom view of an exemplary implant in accordance with the invention.

FIG. 4 illustrates an exemplary implant in accordance with the invention.

FIG. 5 illustrates an exemplary implant in accordance with the invention.

FIG. 6 illustrates an exemplary implant in accordance with the invention.

FIG. 7 illustrates exemplary apparatus and methods in accordance with the invention.

### DESCRIPTION

A human heart includes a left atrium, left ventricle, right atrium and right ventricle. During diastole, the heart muscle relaxes and each ventricle fill with blood from the corresponding atrium. During systole, the ventricles contract, pumping blood out of the ventricles. Blood pumped out of the right ventricle goes through the Right Ventricular Outflow Tract (“RVOT”) into the main pulmonary artery. Blood pumped out of the left ventricle goes through the Left Ventricular Outflow Tract (“LVOT”) into the aorta.

Apparatus and methods for heart valve replacements that may reduce or eliminate obstruction of blood flow through a heart outflow tract during systole are provided. The apparatus may include an implant for supporting a valve in a native annulus of a side of a heart. The apparatus may include an implant for implanting in an atrium of the heart. The implant may be collapsible for deployment into the atrium and thereafter expandable such that the implant is anchorable within the atrium. The implant, when implanted in a side of the heart, may deform during systole to support normal blood through an outflow tract.

The implant may comprise an outer stent, an inner valve support and a transition section spanning between the outer stent and the inner valve support. The implant may be a unitary structure. The unitary structure may be formed from a laser-cut tube. The implant may be formed from two or more attached structures.

The outer stent may include struts. The inner valve support may include struts. The transition section may include struts. The struts may form cells.

Outer stent struts may have a first thickness. Transition section struts may have a second thickness. Inner valve support struts may have a third thickness. The first, second and third thicknesses may be the same. The transition section may include struts having a wall thickness that is thinner than struts of outer stent cells and/or inner valve support cells. The first thickness may be greater than the second thickness and/or the third thickness. The second thickness may be greater than the first thickness and/or the third thickness. The third thickness may be greater than the first thickness and/or the second thickness.

The outer stent may be an expandable prosthetic frame for deployment within an atrium of a heart. The outer stent may be sized to be larger than the atrium. The outer stent may include a cuff at a bottom portion of the implant for extending into an annulus positioned between the atrium and a ventricle of the heart. The cuff may be a cuff that does not anchor the implant in the annulus. The cuff may be an annular portion of the outer stent. The cuff may be referred to herein as an annular ring.

The cuff may not extend into the ventricle. The cuff may extend into the ventricle.

The cuff may be positioned at a bottom of the outer stent. A portion of the outer stent may extend past the cuff.

The inner valve support may be unitary with the outer stent and the transition section. The inner valve support may be positioned within the outer stent. One or more prosthetic leaflets may be attached to the inner valve support. A transition section may span between the outer stent and the inner valve support. The transition section may connect the outer stent to the inner valve support.

The implant may include the outer stent, the inner valve support, the transition section and the cuff. The implant may include one, two, three or four sections. Each of the outer stent, the inner valve support, the transition section or the cuff may comprise a section of the implant.

When implanted in the atrium, a top portion of the implant may contact an atrial roof and the cuff at the bottom of the implant may be positioned in the annulus. The cuff may include the transition section. The cuff may be the transition section. When the implant is implanted in the atrium, the transition section may contact the annulus and position the inner valve support within the outer stent and spaced apart from the annulus.

The apparatus and methods may include positioning the implant in a left atrium such that an inflow end of the inner valve support is positioned a distance away from an aortic root relative to an outflow end of the inner valve support.

The transition section may be deformable. The transition section may be deformable for absorbing force exerted by the annulus on the transition section during systole. The deformation may be elastic. The deformation may include a collapse of cells. The deformation may include bending of cell struts. The deformation may include buckling of cell struts. The deformation may include compression of material.

Deformation of the transition section may enable the implant to mimic deformation of the annulus during systole so that blood flowing through the LVOT follows a normal trajectory. A normal trajectory may be a trajectory of blood, flowing out of the ventricle during systole, in a healthy heart. Deformation of the transition section may reduce deformation of the valve support during systole.

The transition section may deform during systole. The transition section may deform in response to ventricular pressure transferred to implant during systole. The deformation of the transition section may reduce a volume occupied by the cuff. The deformation of the transition section may move the cuff away from an outflow tract of the side of the heart. In a right side of the heart, the deformation may move the cuff to the right, away from the RVOT. In a left side of the heart, the deformation may move the cuff to the left, away from the LVOT. The deformation of the transition section may reduce the volume occupied by the cuff and permit movement of the cuff away from the outflow tract of the side of the heart.

Deformation of the transition section may de-couple the cuff from the inner valve support. Deformation of the transition section may reduce the hoop strength of the cuff. The cuff may absorb ventricular systolic force. The cuff may absorb ventricular systolic force that would have been transferred to the valve support if the transition section did not deform. This may support greater absorption of the systolic force by the cuff.

The deformation of the transition section may alter a profile of the bottom portion of the implant during systole. The implant may conform to a first profile during diastole and to a second profile during systole. The second profile may reduce interference of the implant with blood flowing through an outflow tract. The second profile may reduce interference of the implant with the blood flow relative to interference that would be created if the implant conformed to the first profile during systole. The second profile may reduce interference of the implant with the blood flow relative to the interference that would be created if the implant was in a non-stressed state during systole.

The implant may be deformable. The boss may be deformable. The transition section may be deformable. The transition section may deform, in response to an applied pressure, more than one or more of the implant, boss and transition section would deform.

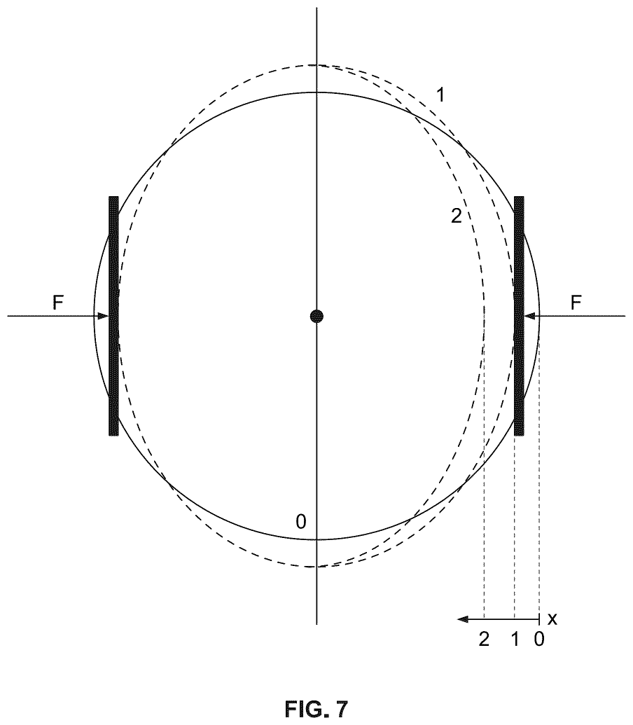

FIG. 7 shows schematically illustrative deformation of the implant. FIG. 7 shows a cross section of implant 0, implant 1 and implant 2. Implant 0 may be at equilibrium. Implant 0 may include a circular cross section when at equilibrium. Implant 0 may have no forces acting on it. Implant 0 may be at equilibrium at X=0. Implant 0 may have a first side and a second side. The first side may be to the right of bisector line 701. The second side may be to the left of bisector line 701. The first side may have the same strength as the second side. Bisector line 701 may be in the middle of the implant. Bisector line 701 may be to the left of center. Bisector line 701 may be to the right of center.

Implant 1 may have force F acting on it. When force F acts on implant 1, implant 1 may deform uniformly. Force F may cause implant 1 to deform to X=1. Force F may be caused by systole. Force F may be caused by a laboratory device. Implant 1 may have a first side and a second side. The first side may be to the right of bisector line 701. The second side may be to the left of bisector line 701. The first side may have the same strength as the second side.

Implant 2 may have force F acting on it. When force F acts on implant 2, implant 2 may deform non-uniformly. Implant 2 may have a first side and a second side. The first side may be to the right of bisector line 701. The second side may be to the left of bisector line 701. Bisector line 701 may be in the center of implant 2. Bisector line 701 may be to the left or right of center. Implant 2 may deform non-uniformly when the first side is softer than the second side. Implant 2 may deform non-uniformly when the first side has decreased strength with respect to the second side. Force F may cause the first side of implant 2 to deform to X=2. Force F may cause the second side of implant 2 to deform similar to the second side of implant 1.

The implant may define a first side and a second side. The first side may be to the left of a plane transecting the implant. The second side may be to the right of the plane. The plane may divide the implant symmetrically. The plane may divide the implant asymmetrically. The plane may transect the implant at any angle relative to an implant central axis. The first side may have increased strength relative to the second side. Increasing the strength of a side may cause that side to experience less deformation under stress. The increased strength of the first side may cause the second side to deform first under stress. Methods may include placing the implant in a left ventricle such that the second side is positioned against the LVOT. In some embodiments, deformation of the implant during systole may facilitate blood flow in the normal trajectory. The deformation may move the implant away from the LVOT during systole to prevent obstruction of the LVOT. The first side may be stronger than the second side by a percentage amount, such as 5%, 10%, 15%, 20%, 25%, 30%, 35%, 40%, 45%, 50% or any other suitable value.

The first side may be a first section. The first section may be a first heart tissue contacting section. The second side may be a second section. The second section may be a second heart tissue contacting section.

The transition section may be formed using one or more of apparatus and methods detailed herein in any suitable manner.

The transition section may include a mechanical buffer material. The mechanical buffer material may include hydrogel.

The transition section may include hydrogel.

The hydrogel may expand when exposed to blood. The hydrogel may absorb shock applied to the implant by a contraction of the heart during systole. The hydrogel may dissipate energy placed on the implant by the contraction of the heart during systole. The hydrogel may prevent the implant from deforming during systole. A first section of the implant may be weaker than a second section of the implant. The hydrogel may be applied to the weaker section of the implant. The hydrogel may protect the weaker section of the implant from structural failure.

The hydrogel may be applied to an outer portion of the transition section. The hydrogel may be applied to an inner portion of the transition section. The hydrogel may replace the transition section. The hydrogel may be encapsulated. The hydrogel may be added before implantation. The hydrogel may be added after implantation.

A section of the implant different from the transition section may include hydrogel as described above in relation to the transition section.

The transition section may include fabric. The fabric may be woven. The fabric may be nonwoven.

The transition section may include any suitable material.

The transition section may include material more compliant than outer stent material and/or inner valve support material.

The transition section may include material thinner than outer stent material and/or inner valve support material.

The transition section may include a pre-bent shape.

The transition section may include a spring.

The transition section may include serpentine struts positioned along a bottom portion of the implant.

The transition section may include serpentine struts positioned perpendicular to a bottom portion of the implant.

The apparatus and methods may include positioning the implant in a heart chamber such that a central axis of the implant is oblique to a central axis of the annulus. The central axis of the implant may be positioned so that, during systole, blood flows through the annulus and away from the LVOT.

The inner valve support may be positioned at an angle relative to the outer stent.

A central axis of the inner valve support may be oblique to a central axis of the outer stent. A central axis of the inner valve support may be oblique to a central axis of the implant.

A first area of a section of the implant may include more struts per volume than a second area of the section of the implant. A first area of a first section of the implant may include more struts per volume than a second area of a second section of the implant. More struts per volume may increase a strength of the first area relative to a strength of the second area.

A first area of a first section of the implant may define a first pattern. A second area of the first section of the implant may define a second pattern. A second area of a second section of the implant may define the second pattern. The first pattern may impart greater strength relative to the second pattern. The pattern may be defined by one or more of an angular positioning of struts, location of overlap between struts, location of connection to other struts and/or any other suitable patterns.

A first area of a section of the implant may include struts with a greater length, width, height and/or thickness relative to struts of a second area of the section. A first area of a first section of the implant may include struts with a greater length, width, height and/or thickness relative to struts of a second area of a second section. The struts with the greater length, width, height and/or thickness may impart a greater strength to the first area. Methods may include positioning the second area adjacent to the LVOT.

A first area of a section of the implant may be formed from material imparting greater strength relative to material used to form a second area of the section. A first area of a first section of the implant may be formed from material imparting greater strength relative to material used to form a second area of a second section. Illustrative materials may include nitinol, fabric, hydrogel and/or any other suitable material. The first area and the second area may be formed from the same material but with a different stiffnesses.

The transition section may include fabric and/or tissue.

The transition section may be formed from fabric and/or tissue. A transition section formed from fabric may load easily into a catheter.

The fabric may include PET, PTFE or any other suitable fabric. The fabric may be flexible. The fabric may be woven. The fabric may be non-woven.

The transition section may be connected to the outer stent and inner stent via suturing or rope.

A transition section formed from fabric, rope or sutures may enable the outer stent to move with respect to the inner stent without placing stress on the inner stent. This may prevent movement of the inner stent while the outer stent is moving. This may prevent the inner stent from being compressed by the transition section during systole.

A transition section formed from fabric may be formed from a one-piece sheet. The one-piece sheet may be connected to the outer stent at a first side. The one-piece sheet may be connected to the inner stent at a second side opposite the first side. The one-piece sheet may be sutured to the outer and inner stents via the first and second sides respectively.

A deformable tube may be connected to an inner or outer surface of the outer stent. The tube may be connected to an inner or outer surface of the transition section. The tube may be connected to an inner or outer surface of the annular ring. There may be a gap between the inner valve support and the annular ring. The tube may be disposed in the gap. The tube may provide support to the inner valve support. The tube may provide support to the annular ring. The tube may provide a buffer between the inner valve support and the annular ring. The tube may ensure a minimum distance is maintained between the inner valve support and the annular ring.

The tube may act as a spring. The tube may dissipate energy. The tube may include one or more of hydrogel, rubber, silicone and/or any other suitable material. The tube may be anchored in place. The tube may be anchored by a friction fit around the annular ring. The tube may expand when implanted to create the friction fit. The tube may expand to control a diameter of the annular ring. The tube may expand when hydrogel, included in the tube, is exposed to blood.

Forming the tube on an inner surface of the implant may include encapsulating the tube within the implant. Struts of the implant may encapsulate the tube. The struts may form a lip to encapsulate the tube. The tube may isolate the annular ring. The tube may be formed to fit the natural valve of the heart. The tube may include a first section. The first section may include properties that impart a greater strength relative to a second section. The properties may be those as described in detail above.

The steps of illustrative methods may be performed in an order other than the order shown or described herein. Some embodiments may omit steps shown or described in connection with the illustrative methods. Some embodiments may include steps that are neither shown nor described in connection with the illustrative methods. Illustrative method steps may be combined. For example, one illustrative method may include steps shown in connection with another illustrative method.

Some embodiments may omit features shown or described in connection with the illustrative apparatus. Some embodiments may include features that are neither shown nor described in connection with the illustrative apparatus. Features of illustrative apparatus may be combined. For example, one illustrative embodiment may include features shown in connection with another illustrative embodiment.

Embodiments may involve some or all of the features of the illustrative apparatus or some or all of the steps of the illustrative methods.

Embodiments may include features that are neither shown nor described in connection with the illustrative apparatus. Features of illustrative apparatus may be combined. For example, an illustrative embodiment may include features shown in connection with another illustrative embodiment. It is to be understood that structural, functional and procedural modifications or omissions may be made without departing from the scope and spirit of the present invention.

The illustrative apparatus and methods will now be described with reference to the accompanying Figures, which form a part hereof. It is to be understood that other embodiments may be utilized and that structural, functional and procedural modifications may be made without departing from the scope and spirit of the present disclosure.

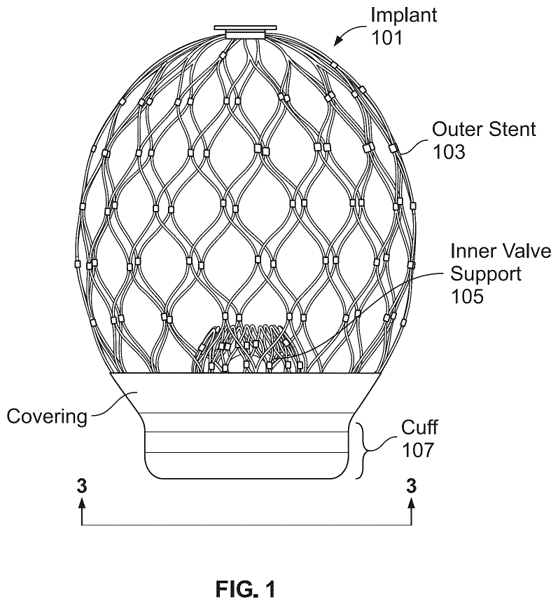

FIG. 1 shows exemplary implant 101. Implant 101 may include outer stent 103 and inner valve support 105. Implant 101 may include cuff 107 near the bottom of the implant that fits within a heart annulus. A covering may extend along some or all of cuff 107. Implant 101 may also include a transition section spanning between inner valve support 105 and outer stent 103.

Implant 101 may include inner valve support 105 pointing upward from the interior of the cuff 107. Inner valve support 105 may be configured to support and retain prosthetic valve leaflets. Inner valve support 105 may direct blood flow from an atrium to a ventricle. Inner valve support 105 may be cylindrical. Inner valve support 105 may be conical or partially conical.

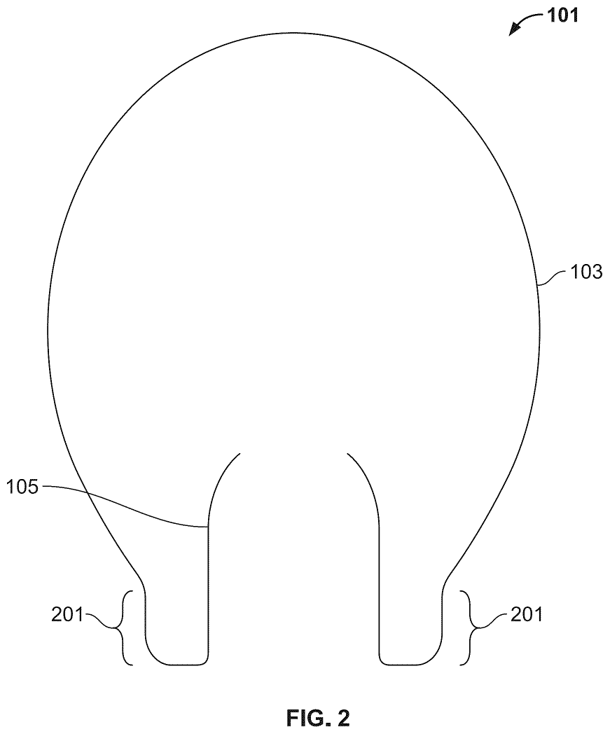

FIG. 2 shows a partial profile of implant 101. Implant 101 may include transition region 201 spanning between outer stent 103 and inner valve support 105. Transition region 201 may include the transition section.

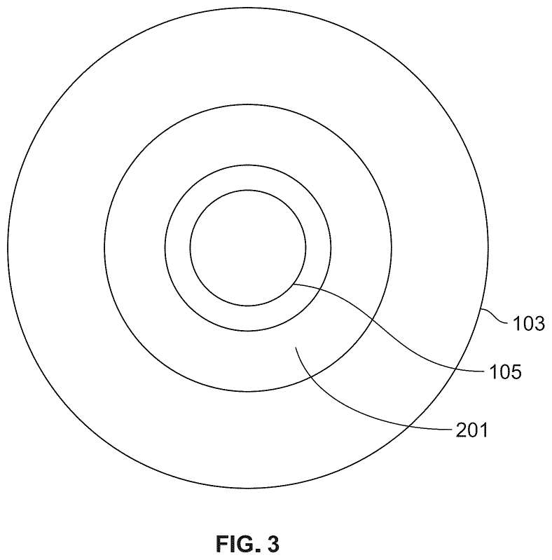

FIG. 3 shows a cross sectional view of implant 101 including outer stent 103, inner valve support 105 and transition region 201.

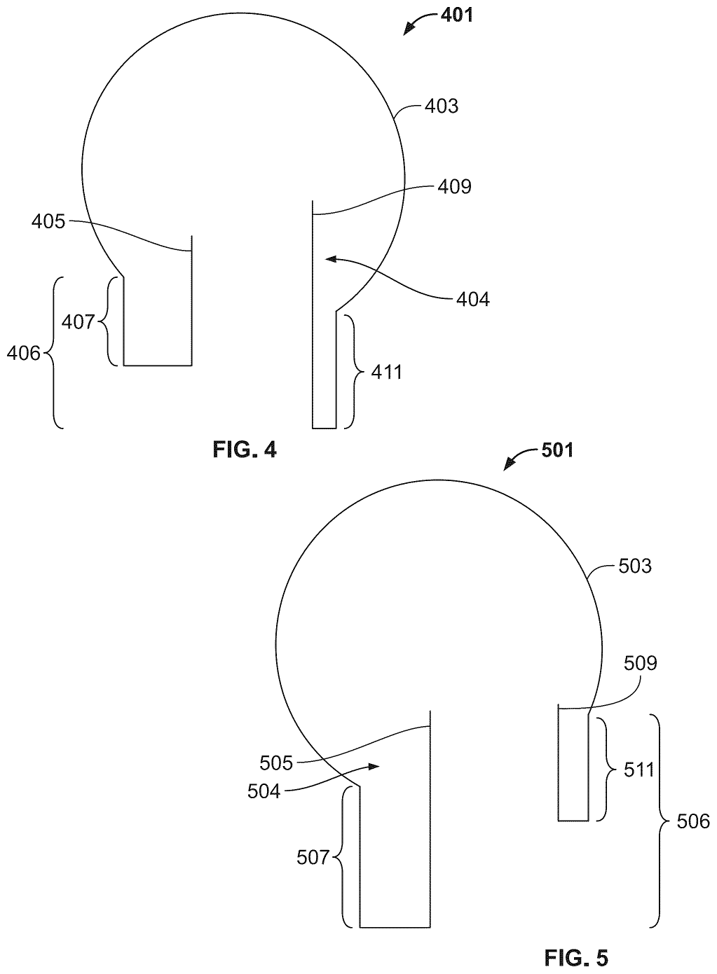

FIG. 4 shows exemplary implant 401. Implant 401 may include outer stent 403 and inner valve support 404. Inner valve support 404 may include anterior section 405 and posterior section 409. Anterior section 405 may have a different size than posterior section 409. Anterior section 405 may be shorter than posterior section 409. Anterior section 405 may be configured to be adjacent to the LVOT. Implant 401 may include cuff 406 near the bottom of the implant. Cuff 406 may be configured to fit within a heart annulus. Cuff 406 may include anterior cuff 407 and posterior cuff 411. Anterior cuff 407 may have a different size and/or shape than posterior cuff 411. Anterior cuff 407 may be wider and/or shorter than posterior cuff 409. A covering may extend along some or all of cuff 406. Implant 401 may also include a transition section spanning between inner valve support 404 and outer stent 403.

Implant 401 may include inner valve support 404 pointing upward from the interior of the cuff 406. Inner valve support 404 may be configured to support and retain prosthetic valve leaflets. Inner valve support 404 may direct blood flow from an atrium to a ventricle. Inner valve support 404 may be cylindrical. Inner valve support 404 may be conical or partially conical.

FIG. 5 shows exemplary implant 501. Implant 501 may include outer stent 503 and inner valve support 504. Inner valve support 504 may include anterior section 505 and posterior section 509. Anterior section 505 may have a different size than posterior section 509. Anterior section 505 may be longer than posterior section 509. Posterior section 509 may be configured to be adjacent to the LVOT. Implant 501 may include cuff 506 near the bottom of the implant that is configured to fit within a heart annulus. Cuff 506 may include anterior cuff 507 and posterior cuff 511. Anterior cuff 507 may have a different size and/or shape than posterior cuff 511. Anterior cuff 507 may be wider and/or taller than posterior cuff 509. A covering may extend along some or all of cuff 506. Implant 501 may also include a transition section spanning between inner valve support 504 and outer stent 503.

Implant 501 may include inner valve support 504 pointing upward from the interior of the cuff 506. Inner valve support 504 may be configured to support and retain prosthetic valve leaflets. Inner valve support 504 may direct blood flow from an atrium to a ventricle. Inner valve support 504 may be cylindrical. Inner valve support 504 may be conical or partially conical.

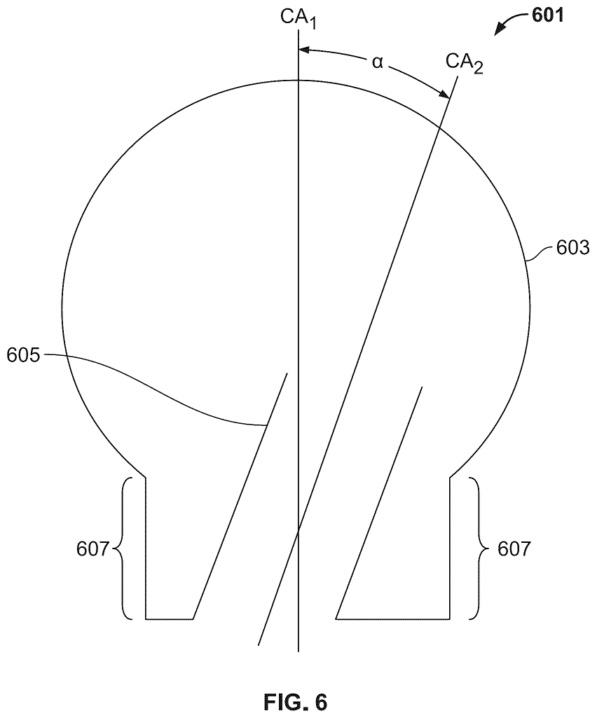

FIG. 6 shows exemplary implant 601. Implant 601 may include outer stent 603 and inner valve support 605. Implant 601 may include cuff 607 near the bottom of the implant that fits within a heart annulus. A covering may extend along some or all of cuff 607. Implant 601 may also include a transition section spanning between inner valve support 605 and outer stent 603. Implant 601 may include central axis CA₁. Valve support 605 may include central axis CA₂. Central axis CA₁ may be oblique to central axis CA₂. Central axis CA₂ may be positioned at an angle α with respect to central axis CA₁. Angle α may be 0, 5, 15, 20, 25, 30, 35 or any suitable number of degrees.

Implant 601 may include inner valve support 605 pointing upward from the interior of the cuff 607. Inner valve support 605 may be configured to support and retain prosthetic valve leaflets. Inner valve support 605 may direct blood flow from an atrium to a ventricle. Inner valve support 605 may be cylindrical. Inner valve support 605 may be conical or partially conical.

All ranges and parameters disclosed herein shall be understood to encompass any and all subranges subsumed therein, every number between the endpoints, and the endpoints. For example, a stated range of “1 to 10” should be considered to include any and all subranges between (and inclusive of) the minimum value of 1 and the maximum value of 10; that is, all subranges beginning with a minimum value of 1 or more (e.g. 1 to 6.1), and ending with a maximum value of 10 or less (e.g., 2.3 to 10.4, 3 to 8, 4 to 7), and finally to each number 1, 2, 3, 4, 5, 6, 7, 8, 9, and 10 contained within the range.

Thus, apparatus and methods for a prosthetic heart valve that maintains natural blood flow have been provided. Persons skilled in the art will appreciate that the present invention may be practiced by other than the described embodiments, which are presented for purposes of illustration rather than of limitation.

What is claimed is:

1. An implant for supporting a valve in a native annulus, the implant comprising:
a first heart tissue contacting section;
a second heart tissue contacting section; and
a mechanical buffer material;
wherein:
the first section is weaker than the second section;
the mechanical buffer material includes hydrogel; and
the hydrogel is disposed on the second section.
